(19) United States
(12) Patent Application Publication
(10) Pub. No.: US 2026/0115422 A1
(43) Pub. Date: Apr. 30, 2026

(54) DEVICES AND METHODS FOR VISUALIZING INTRAVASCULAR PROCEDURES

(71) Applicant: Covidien LP, Mansfield, MA (US)

(72) Inventor: Madeleine A. Roseen, Tustin, CA (US)

(21) Appl. No.: 19/367,334

(22) Filed: Oct. 23, 2025

Related U.S. Application Data
(60) Provisional application No. 63/712,362, filed on Oct. 25, 2024.

(51) Int. Cl. A61M 25/00; A61M 25/06

(52) U.S. Cl. CPC A61M 25/0068; A61M 25/0026; A61M 25/0662; A61M 2025/0037; A61M 2205/32; A61M 2205/3379; A61M 2210/12

(57) ABSTRACT

Devices and methods for visualizing intravascular procedures are disclosed herein. According to some embodiments, the present technology includes a medical device comprising an elongate shaft having a proximal portion and a distal portion configured to be intravascularly positioned at a treatment site within a blood vessel lumen. The medical device can further include a visualization element disposed at the distal portion of the elongate shaft. The visualization element can have a first state in which the visualization element has a first level of radiopacity and a second state in which the visualization element has a second level of radiopacity different than the first level.

### CROSS-REFERENCE TO RELATED APPLICATIONS

This application claims the benefit of and priority to U.S. Provisional Patent Application Ser. No. 63/712,362 filed Oct. 25, 2024, the entire disclosure of which is incorporated by reference herein.

### TECHNICAL FIELD

The present technology relates to devices and methods for visualizing intravascular procedures.

### BACKGROUND

Catheters are essential tools used in a variety of diagnostic and therapeutic procedures, including neurovascular interventions. Accurate placement and navigation of catheters within the body are critical to ensuring the effectiveness of these procedures while minimizing risks to the patient. However, due to the complexity of human anatomy, real-time visualization and guidance are often required to help clinicians maneuver catheters safely to their intended destination. Existing technologies, such as X-ray fluoroscopy, ultrasound, and magnetic resonance imaging (MRI), provide external imaging to track catheter movement. Various solutions have been explored to enhance the visibility of catheters under such external imaging, including the incorporation of radiopaque markers or other materials into or onto the catheter shaft. However, such radiopaque elements, while helpful for guidance of the catheter itself, can make it difficult to distinguish the position of other radiopaque interventional devices advanced through the catheter shaft. Accordingly, there exists a need for improved devices and methods for catheter visualization.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 shows a delivery device in accordance with several embodiments of the present technology. In FIG. 1, a visualization element of the delivery device is shown in an activated state.

FIG. 2 shows a distal portion of the delivery device of FIG. 1 with the visualization element in a de-activated state in accordance with several embodiments of the present technology.

FIG. 3 is an axial cross-sectional view taken along line 3-3 in FIG. 1.

FIG. 4 is an axial cross-sectional view taken along line 4-4 in FIG. 1.

FIG. 5 is a side cross-sectional view of a distal portion of a delivery device in accordance with several embodiments of the present technology.

FIGS. 6A and 6B illustrate a method for using the delivery device in accordance with several embodiments of the present technology.

### DETAILED DESCRIPTION

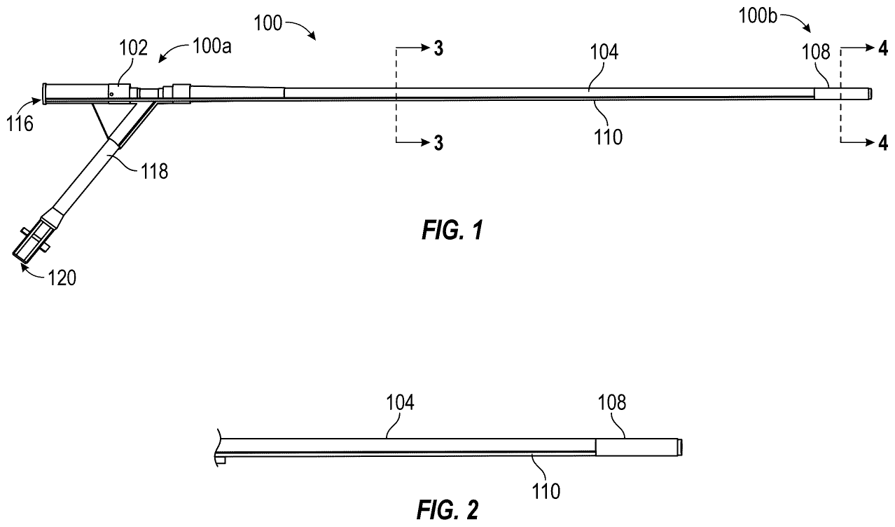

The present technology relates to devices and methods for visualizing intravascular procedures. Some embodiments of the present technology, for example, are directed to delivery devices having visualization elements with selectively variable radiopacity. Specific details of several embodiments of the technology are described below with reference to FIGS. 1-6B.

FIG. 1 shows an example delivery device 100 in accordance with several embodiments of the present technology. As shown in FIG. 1, the delivery device 100 can have a proximal portion 100a configured to be positioned external to the patient and a distal portion 100b configured to be intravascularly positioned within a blood vessel proximate a treatment site. For example, the distal portion 100b can be configured to be positioned within a cerebral vessel proximate an aneurysm or clot material. Other blood vessels and vascular disease states are possible. The delivery device 100 can include a handle 102 at the proximal portion 100a, an elongate shaft 104 extending from a proximal end portion at the handle 102 to a distal end portion coinciding with a distal end portion of the delivery device 100, and a visualization element 108 at the distal portion of the shaft 104.

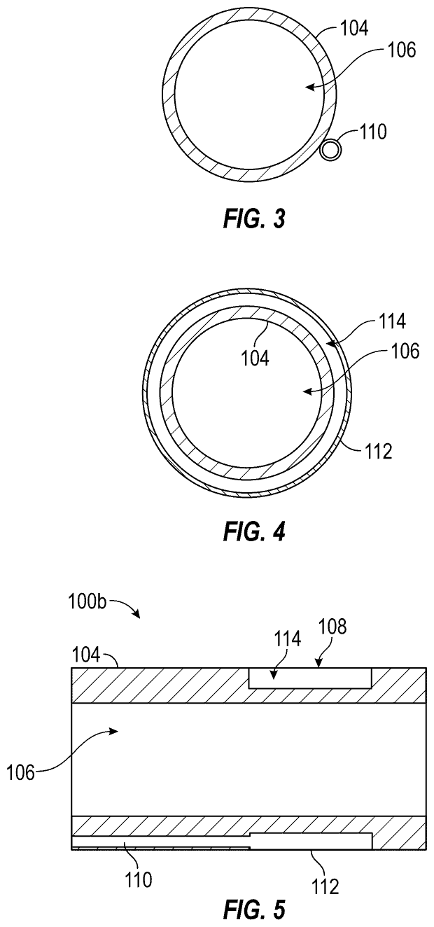

As shown in the axial cross-sectional view of the shaft 104 in FIG. 3, the shaft 104 can comprise a generally tubular sidewall defining a lumen 106 therethrough. The lumen 106 can be sized to receive one or more interventional devices, such as a guidewire, a visualization device, a catheter, an implant (e.g., an intrasaccular device, a flow diverter, coils, etc.) and associated delivery system, a removable treatment device (e.g., a thrombectomy device, etc.) and others.

The visualization element 108 may comprise a radiopaque state (schematically depicted in FIG. 1) for guiding delivery of the distal portion 100b of the device 100 to the treatment site under fluoroscopy, and a non-radiopaque state (schematically depicted in FIG. 1) for better visualizing deployment of a radiopaque interventional device from the distal tip of the device 100. As shown in the axial cross-sectional view of the shaft 104 in FIG. 4, the visualization element 108 can comprise a wall 112 extending around a full circumference of the device 100 and a chamber 114 defined radially between the wall 112 and the shaft 104. The chamber 114 can be configured to receive a radiopaque fluid therein. The chamber 114 can be fluidly coupled to a fluid delivery channel 110 (shown in FIGS. 1-3) that extends from a port 116 at the handle 102 to a distal opening at the chamber 114. The port 116 can be configured to be coupled to a fluid source (not shown), such as a syringe or other means for containing and conveying a radiopaque fluid. The delivery channel 110 can be defined by a tube extending along an outer surface of the shaft 104 (as shown in FIGS. 1-3), a tube disposed within and extending along the lumen 106 of the shaft 104, or a lumen extending through the shaft sidewall (e.g., the lumen is built into the thickness of the sidewall of the shaft 104). Fluid may also be removed from the chamber 114 via the delivery channel 110, for example by coupling a negative pressure source to the port 116. In some embodiments, the delivery channel 110 is configured to be fluidly coupled to an aspiration channel 118 that extends proximally to a port 120 at the proximal portion 100a of the device 100. The port 120 may be configured to be fluidly coupled to a negative pressure source (not shown) for drawing fluid out of the chamber 114 through the delivery channel 110 and through the aspiration channel 118. As discussed in greater detail below, at any time during a given procedure, radiopaque fluid can be delivered to and removed from the chamber 114, thereby increasing and decreasing, respectively, a radiopacity of the visualization element 108.

In some embodiments, the wall 112 comprises a material adhered at its longitudinal ends to an outer surface of the shaft 104 (as shown in FIGS. 1, 2 and 4) and that extends around a full circumference of the shaft 104. In certain examples, the wall 112 extends around less than a full circumference of the shaft 104. In any case, the wall 112 may be oversized relative to an outer diameter of the shaft 104, thereby enabling the delivery of radiopaque fluid between the wall 112 and the shaft 104. In some embodiments, the wall 112 may comprise an elastic material and is sized such that delivery of radiopaque fluid to the chamber 114 causes the wall 112 to stretch and expand radially away from the shaft 104 by a small amount (e.g., no more than a thickness of the sidewall of the shaft 104). Likewise, in such embodiments, removal of fluid (e.g., via the application of negative pressure) causes the wall 112 to radially collapse down onto the shaft 104. In such embodiments where the wall 112 comprises an elastic material, the wall 112 cannot expand beyond a radial thickness (e.g., measured between the wall 112 and the outer surface of the shaft 104) greater than a radial thickness of the sidewall of the shaft 104. Said another way, when expanded, the diameter of the device 100 along the visualization element 108 is increased by less than 50%, less than 40%, less than 30%, less than 20%, or less than 10% of the outer diameter of the shaft 104. This way, the visualization element 108 remains low-profile and does not occlude blood flow.

According to some embodiments, the wall 112 is not elastic and delivery of fluid into the chamber 114 merely fills in dead space between the oversized wall 112 and shaft 104 and does not stretch the material of the wall 112. Still, in such embodiments the visualization element 108 remains low profile, as described above.

FIG. 5 shows another embodiment of a visualization element 108 that is integrated with the shaft 104. The visualization element 108, for example, can comprise an annular chamber 114 defined by an annular groove in the shaft sidewall and a wall 112 extending over the annular groove. In some embodiments, the groove and/or chamber 114 extends around less than a full circumference of the shaft 104. In such embodiments, a radiopaque fluid may be delivered through the delivery channel 110 (which may extend through the sidewall of the shaft 104) to the chamber 114 to increase/decrease the radiopacity of the visualization element 108. When a volume of radiopaque fluid sufficient to fill the chamber 114 is received within the chamber 114, a diameter of the device 100 along the visualization element 108 is substantially the same as the diameter of the device 100 along the shaft 104 immediately adjacent the visualization element 108. In other words, at its maximum radiopacity, the visualization element 108 does not increase a diameter of the device 100. Said yet another way, in some examples the chamber 114 has a fixed volume.

According to certain embodiments, the delivery device 100 includes two or more visualization elements 108 disposed along the shaft 104. In some embodiments, the visualization element 108 can comprise multiple discrete visualization zones that are spaced apart about a circumference of the shaft 104 at substantially the same axial location along the shaft 104. Such circumferential zones may be useful for providing the user with information regarding the rotational position of the shaft 104. For example, at a single axial location, the shaft 104 may have two smaller visualization zones clustered together on one side of the shaft 104, and a single, larger visualization zone diametrically opposed to the two smaller visualization zones, thereby denoting different circumferential positions around the shaft 104. Additionally or alternatively, multiple visualization elements 108 can be spaced apart along the longitudinal axis of the shaft 104 to denote different axial positions along the shaft 104.

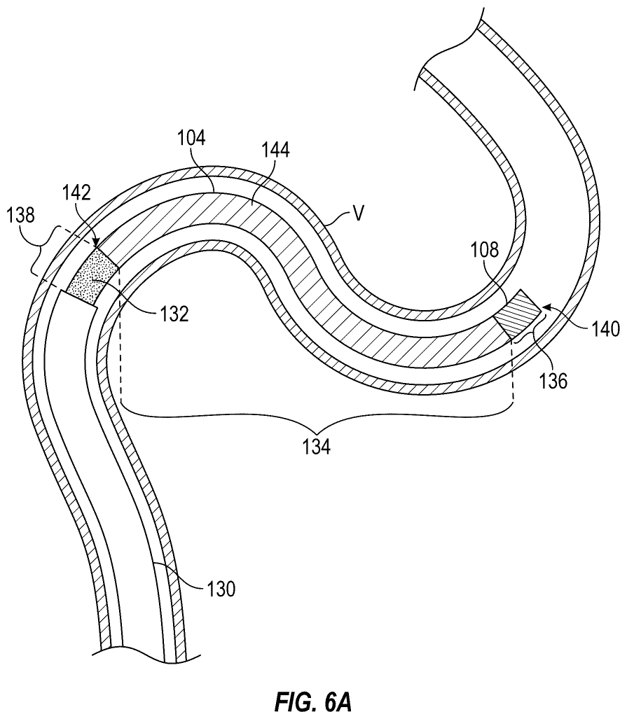

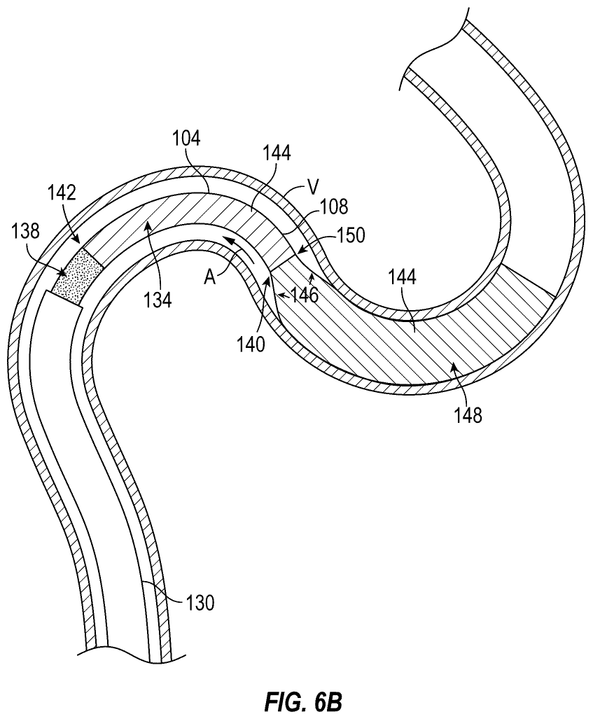

FIGS. 6A and 6B depict a method of deploying an interventional device 144 in a blood vessel V using the delivery device 100 of the present technology. In FIGS. 6A and 6B, the interventional device 144, its associated delivery system, the shaft 104, and the visualization element 108 are depicted as visualized on an x-ray image, e.g., as regions of varying radiopacity. Regions of different radiopaque intensities are denoted by different fill patterns and explained below. The blood vessel V and guide catheter 130 are shown as normal line drawings for the sake of providing a clear environmental reference point.

According to some methods of use, a distal tip 140 of the shaft 104 can be advanced to a treatment site within a blood vessel V. In some embodiments, the shaft 104 is advanced through one or more guide catheters 130 and/or over a guidewire (not shown). Before and/or during advancement of the shaft 104, a radiopaque fluid can be delivered to the chamber 114 of the visualization element 108 such that the visualization element 108 comprises a first level of radiopacity (depicted as region 136) (e.g., where a higher intensity or higher level of radiopacity corresponds to a darker area under fluoroscopy). The first level of radiopacity may enable a user to track movement of the shaft 104 through the vasculature under fluoroscopy.

Once the distal tip 140 of the shaft 104 is positioned at a desired location, the guidewire can be withdrawn and the interventional device 144 can be advanced through the lumen 106 of the shaft 104 to the treatment site. As shown in FIG. 6A, the interventional device 144 can comprise one or more radiopaque materials such that the interventional device 144 comprises a second level of radiopacity (depicted as region 134) under fluoroscopy. The delivery system on which the interventional device 144 is mounted may also comprise a third level of radiopacity (depicted as region 138), which is visible proximal of the interventional device 144. The intensity of the delivery system radiopacity 138 can be less than the compressed interventional device 144 radiopacity (or simply greater than the sheathed or delivery state radiopacity, if the interventional device 144 is not expandable, such as coils) such that the proximal end 142 of the interventional device 144 can be identified/distinguished from the delivery system. The radiopacity level 134 of the compressed (or otherwise sheathed) interventional device 144, however, can appear substantially the same as the radiopacity of the visualization element 108, such that while the interventional device 144 remains within the shaft 104, the interventional device 144 and visualization element 108/distal end of the shaft 104 are virtually indistinguishable under fluoroscopy. This can be problematic when deploying the interventional device 144 in the blood vessel V, as it can be beneficial to know where the proximal end 142 of the interventional device 144 is relative to the distal tip 140 of the shaft 104. For example, the interventional device 144 may be an implant (e.g., a mesh, a braid, etc.) that is configured to expand into apposition with the vessel wall once released. Once in apposition with the vessel wall, the implant's position may become fixed rendering the implant no longer repositionable. Before release of the proximal end 140, however, the implant may still be resheathed and repositioned. As such, knowing precisely when the proximal end 140 of the implant exits the shaft 104 (e.g., the point of no return) allows the user greater control over positioning of the implant, which ensures proper placement for treating the underlying condition.

To address the foregoing challenges, the delivery device 100 of the present technology enables selective radiopacity at the distal end of the shaft 104 via delivery/removal of radiopaque fluid from the visualization element 108. As shown in FIG. 6B, the shaft 104 can be withdrawn proximally (indicated by arrow A) to allow the interventional device 144 to expand into apposition with the vessel wall V.

Prior to or during withdrawal of the shaft 104, some or substantially all of the radiopaque fluid can be withdrawn from the chamber 114 of the visualization element 108, thereby lowering the intensity of the radiopacity of the visualization element 108 and/or eliminating visibility of the visualization element 108 under fluoroscopy. In some cases, a second, substantially non-radiopaque fluid (e.g., saline) can be delivered to the chamber 114 after removal of some or all of the radiopaque fluid. Use of a second fluid may be desirable to avoid having pockets of air along the device 100 while positioned in the vasculature. In some cases, only a portion of the radiopaque fluid is removed and partially or completely replaced with the non-radiopaque fluid, thereby diluting the radiopaque fluid (and lowering the intensity of) without completely emptying the chamber 114. In any of the foregoing examples, the radiopacity of the visualization element 108 can be reduced at least to a level that is less than (e.g., discernibly lighter than) a radiopacity level of the compressed interventional device 144.

With the radiopacity of the visualization element 108 has been reduced and/or substantially eliminated, the location of the distal tip 140 of the shaft 104, however, can still be generally discerned on the fluoroscopic image by visualizing the tapered (in some cases referred to as a “champagne flute”) shape 146 of the newly deployed portion of the interventional device 140. The radiopacity level of the interventional device 144 once expanded (depicted by region 148) can be lower than the radiopacity level of the interventional device 144 in the compressed state within the shaft 104, as the radiopaque material is no longer as densely arranged. As such, the user can visualize withdrawal of the shaft 104 by viewing proximal movement of the interface 150 between the distal end of the darker, higher radiopacity compressed interventional device 144 (distal end of zone 134) and the lighter, lower radiopacity tapered shape 146 of the newly deployed portion of the interventional device 144. As the shaft 104 is withdrawn, the interface 150 approaches the compressed proximal end 142 of the interventional device 144. Once the interface 150 is nearly aligned with but still distal of the proximal end 142, the user may decide whether to complete deployment and fully withdraw the shaft 104 (and thus deploy the interventional device 144) or resheath all or a portion of the interventional device 144 and reposition.

### Examples

The subject technology is illustrated, for example, according to various aspects described herein, including with reference to FIGS. 1-6B. Various examples of aspects of the subject technology are described as numbered examples (1, 2, 3, etc.) for convenience. These are provided as examples and do not limit the subject technology.

Example 1: A medical device, comprising an elongate shaft comprising a proximal portion and a distal portion configured to be intravascularly positioned at a treatment site within a blood vessel lumen; and a visualization element disposed at the distal portion of the elongate shaft, wherein the visualization element has a first state in which the visualization element has a first level of radiopacity and a second state in which the visualization element has a second level of radiopacity different than the first level.

Example 2: The medical device of Example 1, wherein the visualization element comprises a chamber configured to receive a radiopaque fluid therein, and wherein, when the visualization element is in the first state the chamber contains a first volume of fluid and when the visualization element is in the second state the chamber contains a second volume of fluid different than the first volume of fluid.

Example 3: The medical device of Example 2, wherein the first volume of fluid is substantially zero, or wherein the first volume of fluid is non-zero but less than the second volume of fluid, and/or wherein the first volume of fluid is a first volume of a first fluid and the second volume of fluid is a second volume of a second fluid different than the first fluid, the second fluid having a radiopacity greater than a radiopacity of the first fluid.

Example 4: The medical device of Example 2 or Example 3, wherein the second level of radiopacity is greater than the first level of radiopacity.

Example 5: The medical device of any one of the previous Examples, wherein a diameter of the visualization element is substantially the same in the first state as it is in the second state.

Example 6: The medical device of any one of the previous Examples, wherein: the second level of radiopacity is greater than the first level of radiopacity, a diameter of the visualization element in the first state is less than a diameter of the visualization element in the second state, and a diameter of the visualization element in the second state is no more than 50% greater than a diameter of the elongate shaft.

Example 7: The medical device of any one of the previous Examples, further comprising a fluid delivery channel extending from a proximal opening at the proximal portion of the elongate shaft to a distal opening in fluid communication with the visualization element, and wherein the fluid delivery channel is configured to receive a radiopaque fluid therethrough.

Example 8: The medical device of Example 7, wherein the proximal opening of the fluid delivery channel is configured to be fluid coupled to a fluid delivery source.

Example 9: The medical device of any one of the previous Examples, wherein the elongate shaft does not include a radiopaque marker at its distal portion.

Example 10: A method, comprising: advancing a distal portion of an elongate shaft to a treatment site within a lumen of a blood vessel, the distal portion including a visualization element; advancing an interventional device in a delivery state within a lumen of the elongate shaft until the interventional device is aligned with the distal portion of the elongate shaft; withdrawing the elongate shaft relative to the interventional device, thereby deploying at least a portion of the interventional device from the lumen of the elongate shaft; and at a time prior to or during withdrawal of the elongate shaft, reducing a radiopacity of the visualization element relative to a radiopacity of the visualization element during advancement of the elongate shaft to the treatment site.

Example 11: The method of Example 10, wherein reducing the radiopacity of the visualization element includes reducing the radiopacity to a level that enables a sheathed portion of the interventional device be distinguished from the visualization element when viewed under fluoroscopy.

Example 12: The method of Example 11, wherein the level is substantially zero.

Example 13: The method of any one of Examples 10 to 12, wherein reducing the radiopacity comprises removing a radiopaque fluid from the visualization element and/or replacing all or some of the radiopaque fluid with a non-radiopaque fluid and/or a less radiopaque fluid.

Example 14: The method of any one of Examples 10 to 13, wherein the visualization element comprises a material fixed at either longitudinal end to the elongate shaft and a chamber defined between the material and the elongate shaft.

Example 15: The method of Example 14, wherein reducing the radiopacity comprises removing a radiopaque fluid from the chamber.

Example 16: The method of Example 14 or Example 15, wherein the distal portion of the elongate shaft is advanced to the treatment site while radiopaque fluid is contained within the chamber.

Example 17: The method of any one of Examples 10 to 16, further comprising increasing the radiopacity of the visualization element after delivery of the interventional device.

Example 18: The method of Example 17, wherein increasing the radiopacity of the visualization element includes delivering radiopaque fluid to the visualization element.

Example 19: The method of any one of Examples 10 to 18, wherein the elongate shaft does not include a radiopaque marker at its distal portion.

Example 20: A method, comprising: advancing a distal portion of an elongate shaft to a treatment site within a lumen of a blood vessel, the distal portion including a visualization element, wherein the visualization element has a first level of radiopacity during advancement through the blood vessel; advancing an interventional device in a compressed state within a lumen of the elongate shaft until the interventional device is aligned with the distal portion of the elongate shaft; reducing the radiopacity of the visualization element such that the visualization element is no longer discernible under fluoroscopy; and after reducing the radiopacity of the visualization element, withdrawing the elongate shaft relative to the interventional device, thereby allowing the interventional device to expand.

### CONCLUSION

Although many of the embodiments are described above with respect to systems, devices, and methods for delivering implants to cerebral blood vessels, the technology is applicable to other applications and/or other approaches, such as positioning of non-implantable treatment devices within blood vessels and/or treatment of blood vessels outside of the brain. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-6B.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A medical device, comprising:
an elongate shaft comprising a proximal portion and a distal portion configured to be intravascularly positioned at a treatment site within a blood vessel lumen; and
a visualization element disposed at the distal portion of the elongate shaft, wherein the visualization element has a first state in which the visualization element has a first level of radiopacity and a second state in which the visualization element has a second level of radiopacity different than the first level.

2. The medical device of claim 1, wherein the visualization element comprises a chamber configured to receive a radiopaque fluid therein, and wherein, when the visualization element is in the first state the chamber contains a first volume of fluid and when the visualization element is in the second state the chamber contains a second volume of fluid different than the first volume of fluid.

3. The medical device of claim 2, wherein the first volume of fluid is less than the second volume of fluid.

4. The medical device of claim 2, wherein the second level of radiopacity is greater than the first level of radiopacity.

5. The medical device of claim 1, wherein a diameter of the visualization element is substantially the same in the first state as it is in the second state.

6. The medical device of claim 1, wherein:
the second level of radiopacity is greater than the first level of radiopacity,
a diameter of the visualization element in the first state is less than a diameter of the visualization element in the second state, and
a diameter of the visualization element in the second state is no more than 50% greater than a diameter of the elongate shaft.

7. The medical device of claim 1, further comprising a fluid delivery channel extending from a proximal opening at the proximal portion of the elongate shaft to a distal opening in fluid communication with the visualization element, and wherein the fluid delivery channel is configured to receive a radiopaque fluid therethrough.

8. The medical device of claim 7, wherein the proximal opening of the fluid delivery channel is configured to be fluid coupled to a fluid delivery source.

9. The medical device of claim 1, wherein the elongate shaft does not include a radiopaque marker at its distal portion.

10. A method, comprising:
advancing a distal portion of an elongate shaft to a treatment site within a lumen of a blood vessel, the distal portion including a visualization element;
advancing an interventional device in a delivery state within a lumen of the elongate shaft until the interventional device is aligned with the distal portion of the elongate shaft;
withdrawing the elongate shaft relative to the interventional device, thereby deploying at least a portion of the interventional device at the treatment site; and
at a time prior to or during withdrawal of the elongate shaft, reducing a radiopacity of the visualization element relative to a radiopacity of the visualization element during advancement of the elongate shaft to the treatment site.

11. The method of claim 10, wherein reducing the radiopacity of the visualization element includes reducing the radiopacity to a level that enables a sheathed portion of the interventional device be distinguished from the visualization element when viewed under fluoroscopy.

12. The method of claim 11, wherein the level is substantially zero.

13. The method of claim 10, wherein reducing the radiopacity comprises removing a radiopaque fluid from the visualization element.

14. The method of claim 10, wherein the visualization element comprises a material fixed at either longitudinal end to the elongate shaft and a chamber defined between the material and the elongate shaft.

15. The method of claim 14, wherein reducing the radiopacity comprises removing a radiopaque fluid from the chamber.

16. The method of claim 14, wherein the distal portion of the elongate shaft is advanced to the treatment site while radiopaque fluid is contained within the chamber.

17. The method of claim 10, further comprising increasing the radiopacity of the visualization element after the at least partial deployment of the interventional device.

18. The method of claim 17, wherein increasing the radiopacity of the visualization element includes delivering radiopaque fluid to the visualization element.

19. The method of claim 10, wherein the elongate shaft does not include a radiopaque marker at its distal portion.

20. A method, comprising:
advancing a distal portion of an elongate shaft to a treatment site within a lumen of a blood vessel, the distal portion including a visualization element, wherein the visualization element has a first level of radiopacity during advancement through the blood vessel;
advancing an interventional device in a sheathed state within a lumen of the elongate shaft until the interventional device is aligned with the distal portion of the elongate shaft;
reducing the radiopacity of the visualization element such that the visualization element is no longer discernible under fluoroscopy; and
after reducing the radiopacity of the visualization element, withdrawing the elongate shaft relative to the interventional device, thereby allowing the interventional device to at least partially deploy at the treatment site.
